(19) United States
(12) Patent Application Publication
(10) Pub. No.: US 2018/0242884 A1
(43) Pub. Date: Aug. 30, 2018

(54) Sorptive Tab Device for Breath Collection and Analysis

(71) Applicant: Northeastern University, Boston, MA (US)

(72) Inventors: Pushkar Kulkarni, Boston, MA (US); Poguang Wang, Westborough, MA (US); Roger W. Giese, Hanover, MA (US)

(21) Appl. No.: 15/897,602

(22) Filed: Feb. 15, 2018

Related U.S. Application Data
(60) Provisional application No. 62/575,328, filed on Oct. 20, 2017.
(60) Provisional application No. 62/459,573, filed on Feb. 15, 2017.

(51) Int. Cl. A61B 5/097; G01N 33/497; A61B 5/08

(52) U.S. Cl. CPC A61B 5/097; A61B 2010/0087; A61B 5/082; G01N 33/497

(57) ABSTRACT

Disclosed are devices comprising a porous sorptive tab and a substructure, such as a face mask, for collecting breath aerosol from a subject. The device is configured such that, in use, a subject breathes onto or through the sorptive tab. Also disclosed are specific methods of analyzing breath aerosol from a subject.

### CROSS-REFERENCE TO RELATED APPLICATIONS

This application claims the benefit of priority to U.S. Patent Application Ser. No. 62/459,573, filed Feb. 15, 2017; and U.S. Patent Application Ser. No. 62/575,328, filed Oct. 20, 2017.

### GOVERNMENT SUPPORT

This invention was made with government support under Grant No. 5P42-ES017198 from the National Institute of Environmental Health Sciences (NIEHS). The government has certain rights in the invention.

### BACKGROUND

Collection of breath volatiles or aerosol (breath) for clinical diagnosis has been of interest for many years, as has been reviewed (Konstantinidi, E. M., Lappas, A. S., Tzortzi, A. S., Behrakis, P. K., (2015), Exhaled Breath Condensate: Technical and Diagnostic Aspects, Scientific World Journal, 2015, 1-25 doi:10.1155/2015/435160). Because it is more convenient to collect breath aerosols than other biosamples, such as urine or blood, this remains an area of great interest. Nevertheless, aside from the common use of devices for testing alcohol in breath, the numerous devices for collecting breath for analysis of other breath substances have had little or no impact on the practice of clinical diagnostics. Largely, this is because the devices to date are awkward, complicated, nonspecific, or expensive. In a nonspecific test, a researcher doesn't know, with a high degree of certainty, what molecules he or she is measuring. For example, electronic noses are complicated and nonspecific, as has been reviewed (Rock, F., et al. [2008] Electronic Nose: Current Status and Future Trends, Chem. Rev. 108, 705-725). Detection of breath by dog sniffing is also complicated and nonspecific, as has been reviewed (Pleil, J., Giese, R. [2017] Integrating exhaled breath diagnostics by disease-sniffing dogs with instrumental laboratory analysis, J. Breath Research, 11, 032001).

Depending on the particular device for breath analysis, the shortcomings have included: (1) the subject has to stick one end of the device into his or her mouth; (2) the device needs to be connected directly to an inconvenient analyzer, such as a gas chromatograph; (3) the device is useful only for the detection of volatile substances; (4) the device is limited to the detection of only a small number of analytes; (5) the device risks contacting the skin, lips, or saliva of the subject so that substances other than breath may be collected, complicating or even defeating the intended analysis for breath substances; (6) the device does not provide or lead to specific detection; (7) the device only detects known breath substances; (8) the device is not applicable to collecting breath from animals; (9) the device does not readily enable sterilization of collected breath; and (10) the device entails the use of a breath collection bag.

There exists a need for devices and methods for collecting and analyzing breath aerosol in a way that minimizes or overcomes these shortcomings.

### SUMMARY

In certain embodiments, the invention relates to a device comprising a porous sorptive tab removably mounted on a substructure (or mount), wherein the device is configured such that, in use, a subject breathes onto or through the sorptive tab without a need for any part of the device to enter into the mouth of the subject. The substructure holds the sorptive tab.

In certain embodiments, the invention relates to a method comprising the steps of: (1) obtaining a sample of breath aerosol from a subject using any of the devices described herein; and (2) analyzing the sample for the presence of a target analyte. In certain embodiments, the sample is analyzed by mass spectrometry, including cation tag mass spectrometry, nucleic acid hybridization, nucleic acid sequencing, and immunoassay.

In certain embodiments, the invention relates to a method for detecting a disease, a drug, health, a metabolite, a nucleic acid, a protein, a microorganism, a virus, or exposure to an environmental chemical in a subject in need thereof, comprising the steps of: (1) obtaining a sample of breath aerosol from the subject using any of the devices described herein; (2) removing the sorptive tab from the device; and (3) analyzing the sorptive tab for the presence or absence of one or more breath or environmental analytes, wherein the presence or absence of the breath or environmental analyte correlates with the disease, the drug, the health, the metabolite, the nucleic acid, the protein, the microorganism, the virus, or exposure to the environmental chemical.

### BRIEF DESCRIPTION OF THE DRAWINGS

FIG. 1 is a schematic representation of a bubble wand Sorptive Tab Device, comprising a substructure (A and C, which may be one continuous material or separate materials), and a sorptive tab (B) attached to A. One option is to attach just the upper edge of B to A, as with a staple (D).

FIG. 2 is a schematic representation of a Sorptive Tab Device consisting of a sorptive tab (A) with two finger-like extensions (B and C, functioning together as a substructure), where the composition of the entire Sorptive Tab Device (A, B, C) can be the same throughout, or parts B and C can comprise or consist of a different material than part A.

FIG. 3 is a schematic representation of a Sorptive Tab Device consisting of a substructure (A) and sorptive tab (B), where part A comprises or consists of a different material than part B. As in FIG. 1, one option is to attach just the upper edge of B to A, as with a staple (C), as shown.

FIG. 4 is a schematic representation of a Sorptive Tab Device having a sorptive tab (A), stick (B), and staple (C). Other fasteners may be employed to attach the edge of A to B. Part A may be flexible, so that it can bend when breathed upon.

FIG. 5 is a drawing of a Sorptive Tab Device comprising a filter paper stapled to the inside of a face mask, for example, an Isolation Face Mask (NON27122 from MEDLINE).

FIG. 6 is a cartoon of a subject wearing a Sorptive Tab Device comprising a single sorptive tab and isolation mask. A, mask strap; B, sorptive tab; C, mask substructure; and D, staple that attaches B to C. Once the mask is worn, the sorptive tab B can be removed easily from the mask in a non-contaminating way as by using a folded strip of aluminum foil or paper to grab and then remove B from the mask. The sorptive tab can also be removed by grabbing it with nearby parts of the mask, or removing the staple or other device that holds the sorptive tab to the mask. The sorptive tab can then be placed in a metal foil food bag for mailing to a testing laboratory. At the testing laboratory, the sorptive tab can be folded up to facilitate its chemical analysis.

FIG. 7 is a cartoon of a subject wearing a Sorptive Tab Device comprising a sorptive tab, breath mask substructure, and a protection enhancement layer. A, B, and C are the same as in FIG. 6. D is a staple that attaches B and E to C. E is a porous disc enhancement layer that keeps the saliva and tongue of the subject off the sorptive tab. Layer E can also represent a ring that guides the wearer to breathe onto or through the sorptive tab. The sorptive tab B can be recovered by lifting up the protection enhancement layer E, and then employing a folded strip of aluminum foil or paper as described in connection with FIG. 6.

FIG. 8 is a cartoon of a subject wearing a Sorptive Tab Device having a mask substructure, a sorptive tab, and indicating enhancement layer. A, B and C are the same as in FIG. 6. D is a staple that connects B and E to C. E is an enhancement indicating layer containing a substance that changes color when wet, such as Indicating Drierite (comprising calcium sulfate as a drying agent). F is a staple that connects C and E. The purpose of the indicating layer is to reveal, by a color change, that the subject has breathed sufficiently into the mask. This mask can also contain a protection layer as in FIG. 7.

FIG. 9 is a cartoon of a subject wearing a pocketed Sorptive Tab Device. A is the same as in FIG. 6. B is a sorptive pad, such as a plug of cotton. D is part of C that provides a pocket for B. Once the mask is worn, the sorptive tab B can be easily removed from the mask in a non-contaminated way, e.g., by using a folded strip of aluminum foil or paper to grab B and detach it. The foil or paper with B sandwiched in it can then be placed in a metal foil bag for mailing to a testing laboratory. This mask can also contain a protection and/or Indicating layer. Tweezers also can be used to rip the sorptive tab B from the mask.

FIG. 10A is a CAX Mass Tag/MALDI-TOF mass spectrum of a paper filter disc (as a sorptive tab) This is a control experiment; the sorptive tab analyzed was not exposed to breath.

FIG. 10B is a CAX Mass Tag/MALDI-TOF mass spectrum of a paper filter disc after exposure to human breath for 30 minutes inside of a face mask. Two of the more prominent sample peaks have been assigned.

FIG. 11A is a control CAX Mass Tag/MALDI-TOF mass spectrum of a paper filter disc (as a sorptive tab).

FIG. 11B is a CAX Mass Tag/MALDI-TOF mass spectrum of a paper filter disc mounted onto forceps. A subject held the disc with forceps about 2 inches in front of his mouth and breathed onto it for 1 minute, including 10 forced exhalations. Some of the new peaks in FIG. 11B vs. control (FIG. 11A) are designated with arrows.

FIG. 12 is a schematic representation of the chemical reaction between N-(2-(bromomethyl)benzyl)-N,N-diethylethanaminium bromide (CAX-B) and an analyte.

FIG. 13A is a control CAX Mass Tag/MALDI-TOF mass spectrum of a sorptive tab that was cut from a face mask.

FIG. 13B is a CAX Mass Tag/MALDI-TOF mass spectrum of a sorptive tab that was cut from a face mask worn by a subject, displaying many new peaks relative to the control spectrum (FIG. 12A), some of which peaks are designated with arrows and correspond to the compounds shown in Table 1.

### DETAILED DESCRIPTION

### Exemplary Devices

In certain embodiments, the invention relates to a mounted Sorptive Tab Device, wherein the sorptive tab device comprises a porous sorptive tab mounted on a substructure. In certain preferred embodiments, the porous sorptive tab is flexible and can be easily folded by hand. In certain embodiments, the device is configured such that, in use, a subject breathes onto or through the sorptive tab without a need for any part of the Sorptive Tab Device to enter into the mouth of the subject. In certain embodiments, the sorptive tab can be easily removed from the substructure.

In certain embodiments, the invention relates to a method for detecting a disease, a drug, health, a metabolite, a nucleic acid, a protein, a microorganism, a virus, or exposure to an environmental chemical in a subject in need thereof, comprising the steps of: (1) obtaining a sample of breath aerosol from the subject using any of the devices described herein; (2) removing the sorptive tab from the device; and (3) analyzing the sorptive tab for the presence or absence of one or more breath or environmental analytes, wherein the presence or absence of the breath or environmental analyte correlates with the disease, the drug, the health, the metabolite, the nucleic acid, the protein, the microorganism, the virus, or exposure to the environmental chemical. The sorptive tab can be removed, for example, by ripping, cutting, or shearing.

The sorptive tab of a Sorptive Tab Device traps at least part of the breath, especially the aerosol, and especially by adsorption and/or absorption. Breath contains volatiles, semi-volatiles, nonvolatiles, particles, and aerosol, and each of these can be free or combined with the others. The “breath aerosol” is comprised of small liquid particles enriched in semi-volatiles, nonvolatiles, nucleic acids, proteins, microorganisms, viruses, and environmental chemicals from chemical exposure. In certain embodiments, the invention relates to collection of semi-volatile or nonvolatile components.

The Sorptive Tab Device, or just its sorptive tab, can be delivered conveniently after use, as by mail, to a testing laboratory in a metal foil food bag, and at room temperature, especially when the bag contains a substance that prevents microbial growth. Preferred is a testing laboratory utilizing a mass spectrometer, and especially an electrospray mass spectrometer with a linear ion trap or Orbitrap for detection of cation-labeled analytes. The analytes become cation-labeled by reaction with a derivatization reagent containing a cationic group, especially a quaternary amine, and preferably the derivation reagent is an anchimeric-assisted neutral loss tag such as CAX-B.

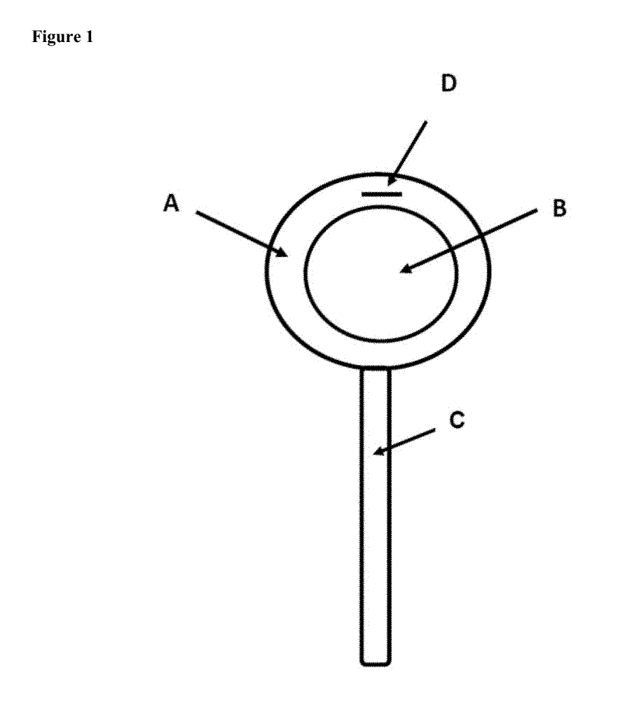

Sorption may also comprise a chemical reaction, a ligand reaction such as an immunoassay, or nucleic acid hybridization. The sorptive tab can be positioned in front of the mouth, under the nose, or both. The substructure both holds the sorptive tab and accomplishes the desired positioning. One option for the sorptive tab is a porous disc of filter paper, which may be chemically or physically modified, with a diameter in the range of about 1-5 cm. One option for a substructure is a bubble wand (bubble wands are ordinarily used for blowing bubbles). The bubble wand substructure can have a single loop to house a single sorptive tab, as illustrated in FIG. 1. The sorptive tab (B) may be attached to the substructure (A) only at the top of the A, for example with a staple (D) as shown in this figure. Such attachment has the advantage that the sorptive tab then is a flap, to reveal when it is impacted by breath and to what degree. This can be important in testing for drugs of abuse, since such subjects will try to defeat the device. Breathing also can be monitored by using a flexible sorptive tab in a bubble wand device, where the sorptive tab moves when breathed upon. Another way to monitor breathing with this kind of device is to install one or more ribbons or strings which are perturbed by breath from a subject, for example attach a ribbon over a small hole in the center of the sorptive tab.

A bubble wand Sorptive Tab Device also can have more than one loop to house multiple sorptive tabs which may be the same or different. When multiple sorptive tabs are present, then different sorptive tabs can be employed having different properties to target different classes of breath substances for collection and analysis. There can be a stack or other array of two or more sorptive tabs which may be the same or different.

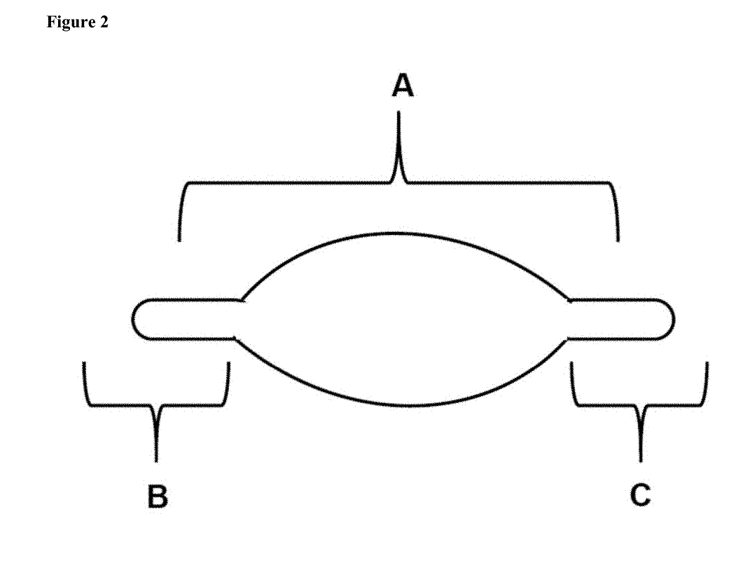

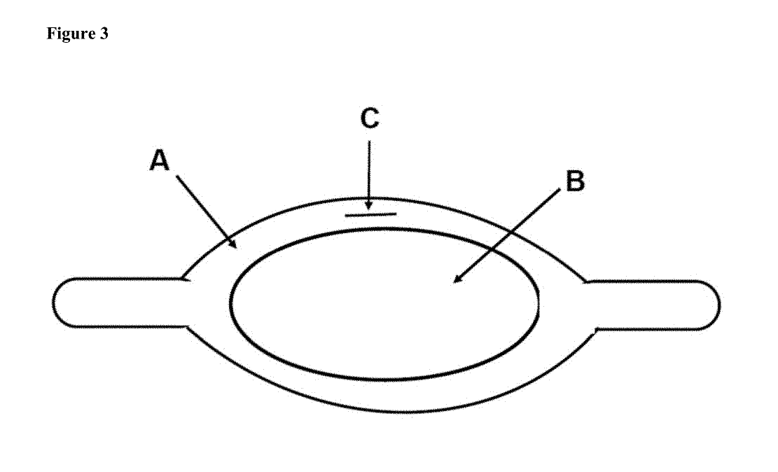

Other substructures can be used such as a wooden, cardboard, paper, or plastic flat stick to which the sorptive tab is attached at one end as by stapling, gluing or welding; a round stick with a slot at one end to mount a sorptive tab: a stick that fits into a hole at the end or edge of a sorptive tab; a stick with a clasp at its end for a sorptive tab, a pin, tweezers, tongs or forceps to hold the sorptive tab. The substructure can be an extension of the sorptive tab, such as two finger-like extensions located diametrically so that the user can hold one finger extension with one hand, and the other finger extension with the other hand, during breath collection. This type of substructure is illustrated in FIG. 2. The finger-like extensions can also consist of, or comprise, another material such as cardboard, metal, wood or plastic. The substructure of a sorptive tab can consist of a loop of wood, cardboard, paper or plastic, which loop mounts the sorptive tab, where the loop has finger-like extensions, as illustrated in FIG. 3. The option of attaching the sorptive tab to the substructure with a staple is illustrated in FIG. 3.

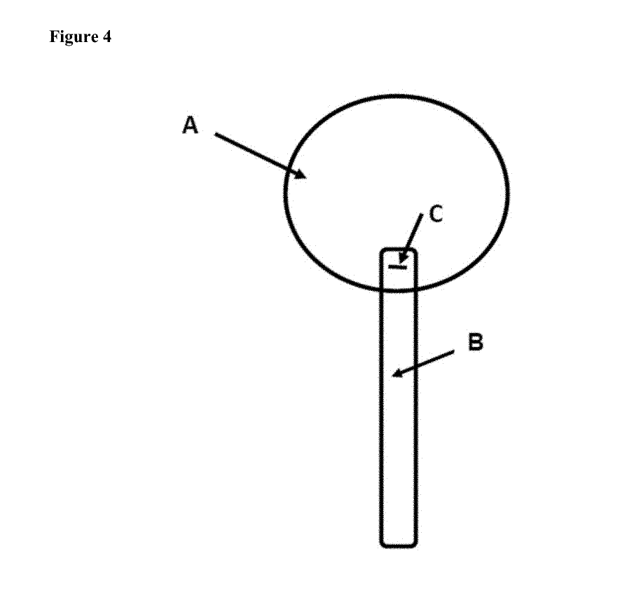

The Sorptive Tab Device can comprise a sorptive tab (to act as a flap), such as a disc of filter paper, attached near its edge, as by stapling, to the end of a stick, such as a flat wooden or plastic stick. This version of a Sorptive Tab Device is illustrated in FIG. 4. As in FIG. 1, here the sorptive tab also can function as a flap to reveal an acceptable level of breathing from a subject. This can be important in testing for drugs of abuse, since the subjects will try to defeat any test for their exposure.

The sorptive tab of the Sorptive Tab Device usually collects breath by providing one or more of the following sorptive mechanisms: hydrophobic, hydrophilic, ion exchange, hydrogen bonding, van der Waals, dipole, and charge-induced dipole. The sorptive tab can have tortuous channels for the air to flow through to provide lots of contact with the breath for efficient sorption. The preferable range of thickness for a sorptive tab is about 0.1 mm to about 1 cm, but larger thickness may be used up to about 10 cm.

The Sorptive Tab Device may have more one or more enhancement layers providing additional sorptive or other properties. The one or more enhancement layers may provide one or more of the following properties or compositions to enhance the performance of the Sorptive Tab Device: (1) Two protective, relatively nonsorptive layers, e.g., of plastic or metal mesh, that sandwich an internal sorptive layer, can be present to avoid contamination of the internal sorptive layer during handling. (2) An indicating, outer layer (on the side away from the face when in use) can be present which contains a moisture indicator such as Indicating Drierite (which changes color from blue to pink when hydrated due to the presence of CoCl₂). This outer layer can establish that the sorptive tab has been exposed adequately to a breath of interest. The CoCl₂ indicator can be incorporated into a variety of materials. Another option for an indicating layer is a layer that contains a pH-sensitive dye which changes color when subjected to the CO2 acidity of breath, and this can be enhanced by including carbonic anhydrase. A pH-indicating dye can be present instead in the sorptive layer. (3) A protective outer layer can be present to minimize contamination of the inner sorptive layer by substances present in the environment in which the Sorptive Tab Device is used. (4) An antimicrobial layer can be present which contains an antimicrobial substance to avoid microbial growth on the sorptive tab (or an antimicrobial substance can be present in the sorptive layer). (5) One or more supplemental, sorptive layers can be present with different sorptive properties toward different substances in breath. (6) One or more strengthening layers may be present to make the sorptive tab slightly more rigid for better collection of breath.

One or more of the following objects, when porous, may constitute or comprise the sorptive tab, or represent the shape or design of the sorptive tab, sometimes in combination, and sometimes as multiples or combination of multiples: sheet, membrane, filter, flap, tissue paper, plug, funnel, spoon, strip, sponge, pillars, cloth, web, column, chromatographic bed, particles, monolith, mesh, disc, block, nest, fibers, woven or nonwoven fiber bed, plate, blade, button, strainer, bottle, basket, ring, dish, tube, bag, pad, ball, film, coil, string, flower, bush, toothbrush, tree, thistle, dish, hat, capsule, pillow, rug, brush, screen, paper, cellulose, cotton, glass, silica, plastic, carbon, metal, salt, nanotubes, ceramic, cracks, spiral, crevices, polymer, gel, polyamide, antibody, molecular-imprinted polymer, layer, aptamer, nucleic acid, protein, ligand, surfactant. The sorptive tab may comprise: sheet, membrane, filter, flap, plug, strip, sponge, cloth, web, monolith, mesh, disc, woven or nonwoven fiber bed, bag or pad. The sorptive tab can be set up as two or more stacked layers, parallel or not, and spaced or not. The sorptive tab can be in motion as by spinning or vibrating or oscillating. The sorptive tab preferably is flexible enough to be easily folded by hand.

In certain embodiments, a routine hospital isolation face mask can be used to collect a sample of breath volatiles or aerosol (breath). In certain embodiments, the invention relates to any of the devices described herein, wherein the substructure is a face mask, such as a hospital isolation face mask or a shop mask. In this case, the sorptive tab can be an inherent part of the mask, or is mounted onto the mask, preferably on the side facing the face of the subject.

In certain embodiments, the invention relates to any of the devices described herein, wherein the sorptive tab provides a consistent and reliable porous material for breath collection. In certain embodiments, the size and composition of the sorptive tab are optimized for breath collection and optimized extraction of analyte. In certain embodiments, the sorptive tab is sterile before it is mounted on the substructure. In certain embodiments, the sorptive tab does not comprise a dye.

In certain embodiments, the invention relates to any of the devices described herein, wherein the sorptive tab, in use, does not come in contact with the subject's hands.

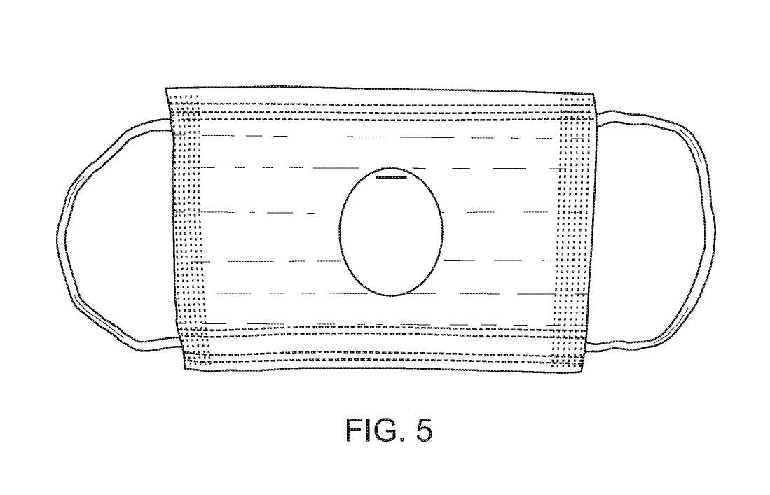

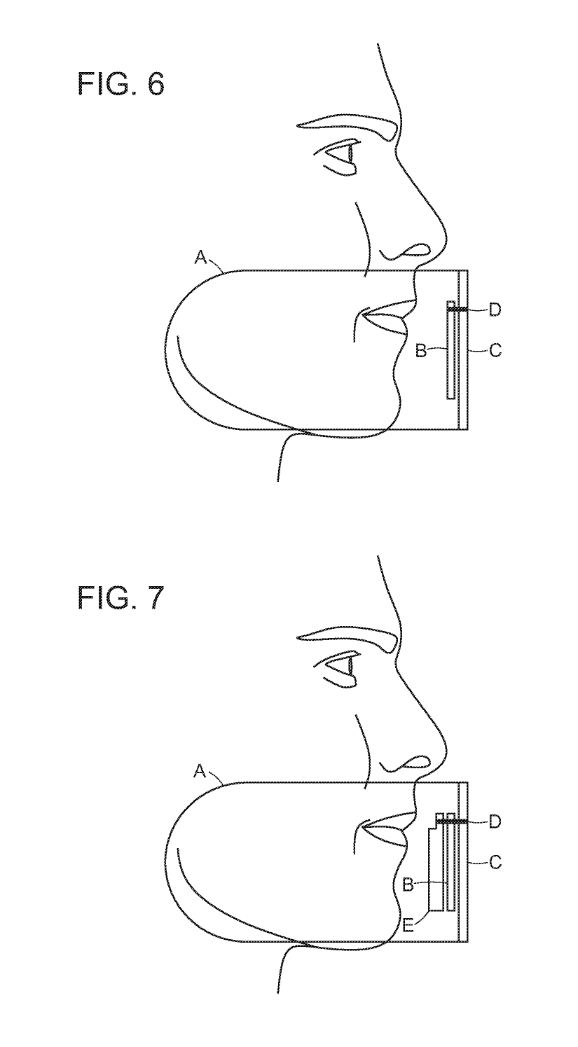

In certain embodiments, the invention relates to any of the devices described herein, wherein the substructure is an isolation mask. In one form of the Sorptive Tab Device, the sorptive tab consists of a small sheet of sorptive material such as filter paper or membrane that is stapled at one of its ends to the mask, as shown in FIG. 5. A cartoon showing a subject wearing a Sorptive Tab Device where the substructure is a mask is shown in FIG. 6. After use, the wearer or another person can tear off the sheet, as with a folded piece of aluminum foil or paper, and put the sheet and foil into a metal foil food bag. The entire Sorptive Tab Device also can be placed in such a bag. The metal foil food bag can contain a drying agent (e.g. bag or porous capsule of desiccant material such as silica to avoid subsequent microbial growth on the collected breath aerosol by drying it). Another kind of anti-microbial agent can be present in the food bag such as one or more of the following: alcohol, acid, volatile solvent, metal, oil, surfactant, antibiotic agent, or antifungal agent. The metal food bag is then sealed by compressing the sealing end, and can be mailed to a testing laboratory for analysis of the breath on the sorptive tab, as by cation mass tag/mass spectrometry (Wang. P., Zhang, Qi, Yao, Y., Giese, R. W. (2015) Cationic Xylene Tag for Increasing Sensitivity in Mass Spectrometry, J. Am. Soc. Mass Spectrom. 26:1713-1721, doi:10.1007/s13361-015-1200-4).

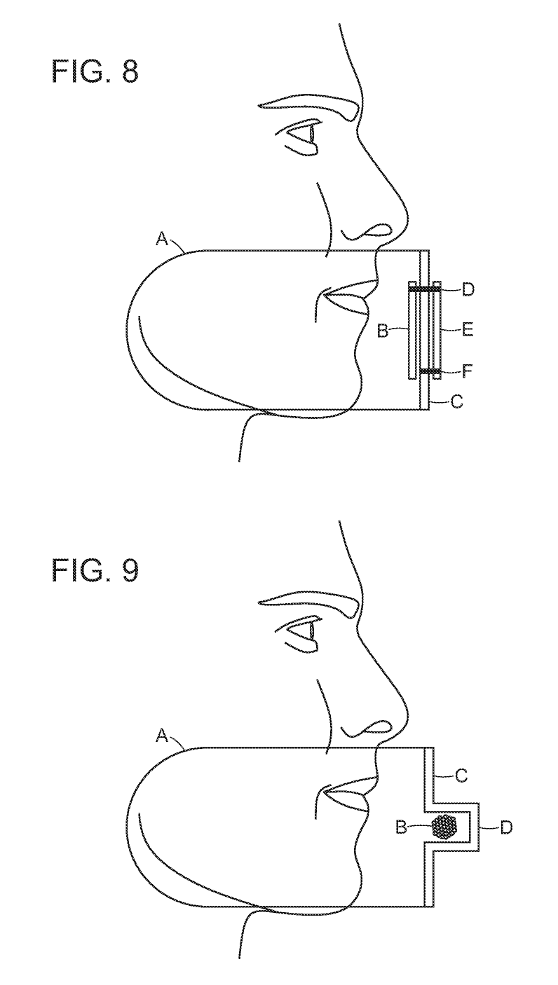

A given sorptive tab, sometimes accompanied by one or more enhancement layers in an isolation mask, may enhance performance in one or more of several ways including: consisting of or comprising an optimum sorptive material for one or more breath components of interest; easy recovery of the sorptive tab(s) from a user-worn mask; high purity and relatively small size to yield an eluted breath sample with a low or zero level of interferences in a subsequent stage of chemical analysis; visual color change upon hydration to establish that a breath mask has been used appropriately; indication of the zone of the mask which is to be targeted by the breath; relatively small size for easy insertion into a small metal-foil food bag; protection of a sorptive tab from contact with saliva as from the tongue of the user; presence of more than one sorptive tab, each of which tends to target different breath substances, or provide an opportunity to store multiple aliquots of breath as repository samples for analysis at a later date; opportunity to fractionate the breath sample into particulate and nonparticulate components by providing a tortuous flow path; opportunity to provide a stiff barrier between the mouth or face and the sorptive material to avoid contamination of the breath sample with facial or saliva substances; improved opportunity to sample nasal breath; or alternatively, prevent exposure of the sorptive tab to the nasal breath. Cartoons showing some options for enhancement tabs are shown in FIGS. 7 and 8. A cartoon illustrating a plug of porous material such as cotton as an sorptive tab is shown in FIG. 9.

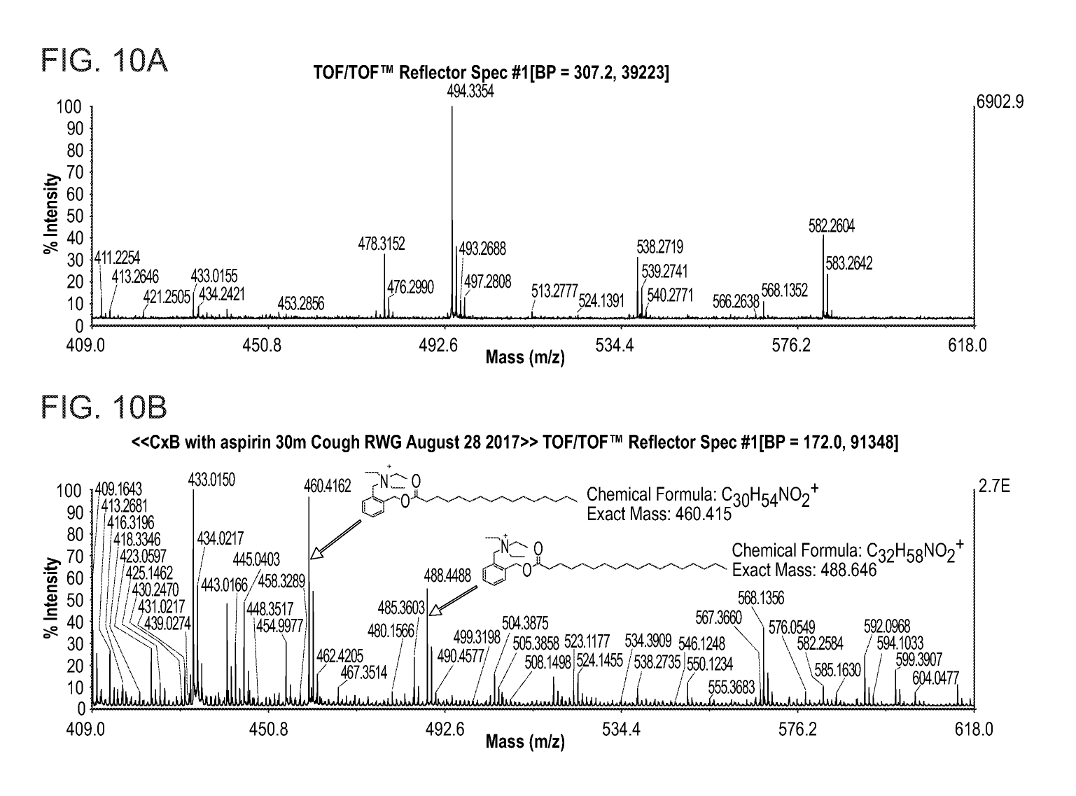

Detection of breath chemicals on sorptive tabs by cation mass tag mass spectrometry is shown in FIGS. 10A, 10B, 11A, 11B, 13A, and 13B.

The sorptive tab of an M-Sorptive Device can have various shapes and sizes to optimize its usefulness. For example, a sorptive tab with a rectangular shape can be set up to collect just the part of the breath at the midpoint of the lips to (analogous to putting one's finger over the mouth to indicate quietness), thereby avoiding interference with breathing, when a mask mount is employed. A sorptive tab with a sharp tip can allow the elution solvent in a later step to drip readily into a receiving test tube or vial. The sorptive tab itself can be set up or flap on the outer face of the isolation mask as by having a hole in the mask and stapling one edge of the sorptive tab so that it covers this hole and flaps out when breath from the subject flows through the hole.

The sorptive tab(s) and enhancement layers can be mounted onto an isolation mask in several ways, such as the following: stapling, sewing, gluing, welding, clamping, melting, taping, physical trapping, pocketing, cross-linking, and Velcro. The sorptive tab(s) can be an inherent part of a face mask, where the sorptive tab is simply the part that is breathed on or through and then removed, as by cutting or shearing, for chemical analysis.

### Exemplary Methods

In certain embodiments, the invention relates to a method comprising: (1) obtaining a sample of breath aerosol from a subject; and (2) analyzing the sample for the presence or absence of a target analyte.

In certain embodiments, the invention relates to any of the methods described herein, wherein the target analyte is an analyte for detecting a disease, a drug, health, a metabolite, a nucleic acid, a protein, a microorganism, a virus, or exposure to an environmental chemical.

In certain embodiments, the invention relates to any of the methods described herein, wherein the target analyte is a target analyte for disease detection.

In certain embodiments, the invention relates to any of the methods described herein, wherein the target analyte is a target analyte for disease exclusion.

In certain embodiments, the invention relates to any of the methods described herein, wherein the sample of breath aerosol is obtained using any one of the devices described herein. In certain embodiments, the invention relates to any of the methods described herein, wherein the sample of breath aerosol is obtained by exposing a sorptive tab to breath aerosol from the subject.

In certain embodiments, the invention relates to any of the methods described herein, wherein the sorptive tab is mounted on the inside surface of a face mask, for example, a fabric face mask. In certain embodiments, the face mask is positioned over the nose or mouth of the subject. In certain embodiments, the face mask is positioned for an amount of time from about 1 minute to about 40 minutes, for example, about 20 minutes or about 30 minutes.

In certain embodiments, the invention relates to any of the methods described herein, wherein the sorptive tab is positioned in front of the nose or mouth of the subject. In certain embodiments, the sorptive tab is positioned for an amount of time from about 30 seconds to about 10 minutes, for example, about 1 minute.

In certain embodiments, the invention relates to any of the methods described herein, wherein the method further comprises the step of: cleaning the face mask before obtaining the sample.

In certain embodiments, the invention relates to any of the methods described herein, wherein the sample is analyzed by mass spectrometry, for example cation mass tag/mass spectrometry (Wang. P., Zhang, Qi, Yao, Y., Giese, R. W. (2015) Cationic Xylene Tag for Increasing Sensitivity in Mass Spectrometry, J. Am. Soc. Mass Spectrom. 26:1713-1721, doi:10.1007/s13361-015-1200-4).

In certain embodiments, the invention relates to any of the methods described herein, wherein the sample is analyzed by mass spectrometry. In certain embodiments, liquid chromatography-electrospray ionization-tandem mass spectrometry (LC-ESI-MS) is employed in the analysis. In certain embodiments, gas chromatography-mass spectrometry (GC-MS) is employed in the analysis.

In certain embodiments, the invention relates to any of the methods described herein, wherein the target analyte is non-volatile. In certain embodiments, the invention relates to any of the methods described herein, wherein the target analyte is semi-volatile.

In certain embodiments, the invention relates to any of the methods described herein, wherein the disease is cancer.

### EXEMPLIFICATION

### Example 1

### Analysis of a Face Mask-Mounted Tab After 2-Propanol Extraction

A sorptive tab (Fisher brand Filter Paper Cat. No. 09-803-6A, 4.25 cm) was mounted inside of a hospital isolation face mask with a staple. A human subject wore the mask for 30 minutes, including providing 30 huffs. Then, the sorptive tab was removed from the mask; and the sorptive tab was soaked in 5 mL isopropanol for 2 h. The solvent was removed under vacuum followed by addition of 200 μL of 50% ACN (Solution C). 2 μL of Solution A (N-[2-(bromomethyl)benzyl]-N,N-diethylethanaminium bromide (CAX-B) (2.4 mg/mL in 50% ACN))+10 μL of Solution B (triethylamine (100 μL/mL in 50% ACN)) were added to 200 μL of Solution C in a vial and allowed to stir for 12 h at room temp. 2 μL from above vial was diluted in 400 μL of 5% α-cyano-4-hydroxycinnamic acid (CCA) matrix and 0.55 μL loaded per spot on MALDI-TOF plate and a MALDI-TOF-MS spectrum was obtained.

Many new peaks were observed (compare FIG. 10B with FIG. 10A, where FIG. 10A is the control (sorptive tab not exposed to breath)). Two peaks were identified as CAX-labeled fatty acids.

### Example 2

### Analysis of a Forceps-Mounted Tab After 2-Propanol Extraction

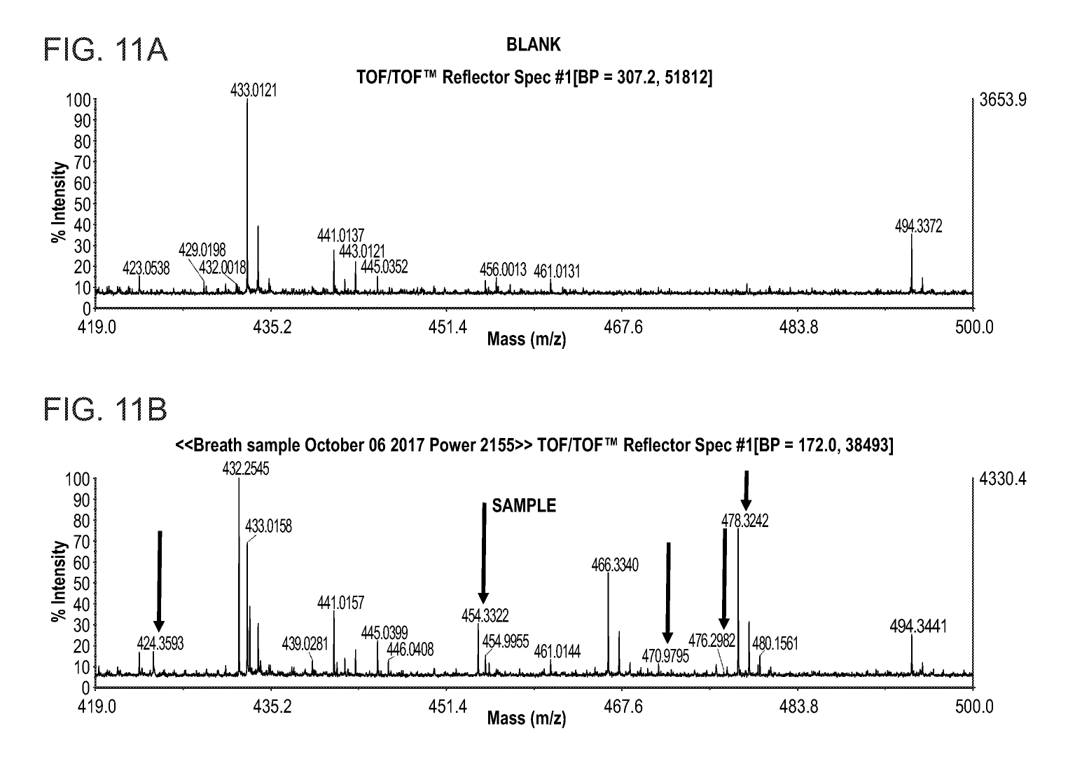

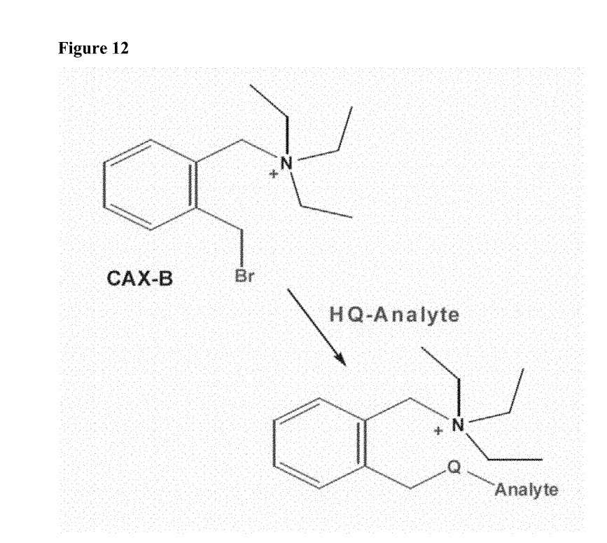

A sorptive tab (Fisher brand Filter Paper Cat. No. 09-803-6A, 4.25 cm) was held with forceps about 2 inches in front of a subject's mouth for 1 minute, including 10 forced exhalations. Then, the sorptive tab was soaked in 5 mL isopropanol for 2 h. The solvent was removed under vacuum followed by addition of 200 μL of 50% ACN (Solution C). 2 μL of Solution A (CAX-B (2.4 mg/mL in 50% ACN))+10 μL of Solution B (triethylamine (100 μL/mL in 50% ACN)) were added to 200 μL of Solution C in a vial and allowed to stir for 12 h at room temp. 2 μL from above vial was diluted in 400 μL of 5% CCA matrix and 0.55 μL loaded per spot on MALDI-TOF plate and a MALDI-TOF-MS spectrum was obtained. Many new peaks were observed (compare FIG. 11B with FIG. 11A, wherein FIG. 11A is the control).

### Example 3

### Analysis of an Inherent Sorptive Tab from a Face Mask After 2-Propanol Extraction

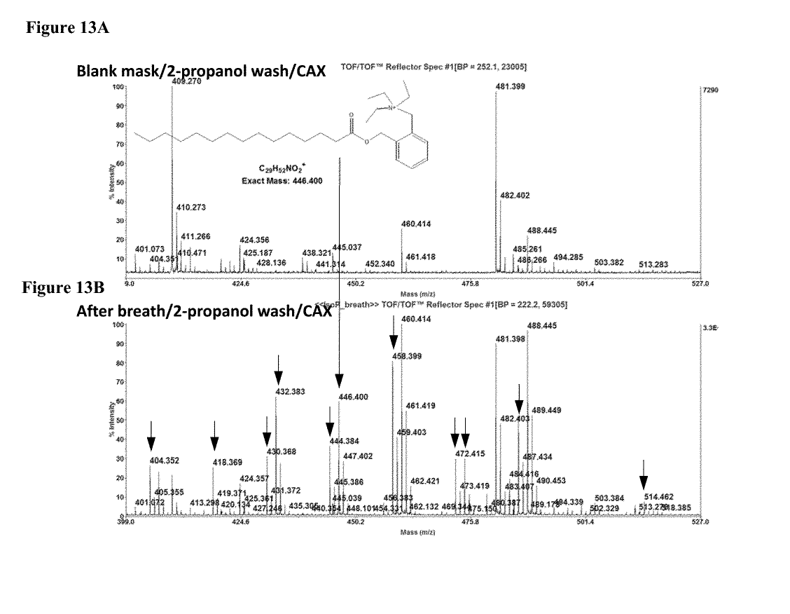

An isolation face mask (NON27122, EN14683 Type II from Medline) was washed with 5×1 mL of 2-propanol and air dried. It was then worn by a subject who breathed normally into it for 20 minutes. The sorptive tab (1.5 inch square region exposed to the breath) was cut out and eluted with two portions of 1 mL of 2-propanol which were combined and evaporated to dryness. 20 μL of 1 mg/mL of CAX-B in 50% acetonitrile containing 10 μL/mL of triethylamine was added and the sample was kept at 38° C. for 14 hours. 1 μL was combined with 400 μL of 5% CCA matrix and 0.55 μL was loaded per spot on the MALDI plate and a MALDI-TOF-MS spectrum was obtained, giving the data shown in FIG. 13B, where peaks for several fatty acids are seen along with many peaks not present in the control sample (FIG. 13A) derived similarly from a sorptive tab not exposed to breath. The fatty acids observed are presented in Table 1.

TABLE 1
Assignment of peaks to fatty acids, assuming linear chain isomers.

| C | Monounsaturated | Saturated |
|---|---|---|
| 12 | | C₂₆H₄₆NO₂⁺ |
| | | Exact Mass: 404.353 |
| 13 | C₂₇H₄₆NO₂⁺ | C₂₇H₄₈NO₂⁺ |
| | Exact Mass: 416.353 | Exact Mass: 418.369 |
| 14 | C₂₈H₄₈NO₂⁺ | C₂₈H₅₀NO₂⁺ |
| | Exact Mass: 430.369 | Exact Mass: 432.384 |
| 15 | C₃₀H₅₂NO₂⁺ | C₂₉H₅₂NO₂⁺ |
| | Exact Mass: 458.400 | Exact Mass: 446.400.415 |
| 16 | C₃₁H₅₄NO₂⁺ | C₃₀H₅₄NO₂⁺ |
| | Exact Mass: 472.415 | Exact Mass: 460.415 |
| 17 | C₃₂H₅₆NO₂⁺ | C₃₁H₅₆NO₂⁺ |
| | Exact Mass: 486.431 | Exact Mass: 474.431 |
| 18 | | C₃₂H₅₈NO₂⁺ |
| | | Exact Mass: 488.447 |

### Example 4

### Analysis of a Forceps-Mounted Sorptive Tab by Nucleic Acid Hybridization or Sequencing

A sorptive tab obtained as in Example 3 is extracted with buffer and the resulting solution is subjected to amplification by the polymerase chain reaction, which may include signal-labeled probes. The resulting sample is tested using a commercial hybridization chip for DNA sequences of interest, or by next generation DNA sequencing. Alternatively, when the sorptive tab is substituted with a nucleic acid, the sorptive tab, after exposure to breath, can be washed and subject to a DNA probe for a nucleic acid sequence of interest, where the probe contains a signal group for detection such as a fluorophore (fluorescent molecule), a chemilumiphore or biotin, where the latter is detected by using streptavidin or avidin attached to a signal group such as a fluorophore a chemilumiphore, or an enzyme. Alternatively, the streptavidin or avidin can be reacted in turn with a biotin-labeled enzyme.

### Example 5

### Analysis of a Forceps-Mounted Sorptive Tab by an Immunoassay

A sorptive tab obtained as in Example 3 is extracted with buffer and the resulting solution is subjected to an immunoassay for a protein, a microorganism, or a drug. Alternatively, when a protein or microorganism of interest is attached firmly to the absorptive tag, as by having a corresponding antibody for it covalently bonded to the sorptive tab, then the sorptive tab can be subjected directly to an immunoassay by incubating it with an second antibody recognizing the protein or microorganism of interest, where the second antibody bears a signal such as a fluorophore a chemilumiphore, an enzyme. The second antibody can also be labeled with biotin which can be detected in turn by adding streptavidin (or avidin) labeled with a signal such as a fluorophore, a chemilumiphore, or an enzyme. Alternatively, the streptavidin or avidin can be reacted in turn with a biotin-labeled enzyme.

### Incorporation by Reference

The contents of any US patents, and US and PCT (designating the US) published patent applications are hereby incorporated by reference in their entirety to the same extent as if each of them was specifically and individually indicated to be incorporated by reference.

### Equivalents

While this invention has been particularly shown and described with references to example embodiments thereof, it will be understood by those skilled in the art that various changes in form and details may be made therein without departing from the scope of the invention encompassed by the appended claims.

We claim:

1. A device comprising a porous sorptive tab and a substructure, wherein the porous sorptive tab is removably mounted on the substructure, and the device is configured such that, in use, a subject breathes onto or through the sorptive tab without a need for any part of the device to enter into the mouth of the subject.

2. The device of claim 1, wherein the sorptive tab is flexible.

3. The device of claim 1, wherein the sorptive tab is foldable.

4. The device of claim 1, wherein the sorptive tab has a diameter in the range of about 1 cm to about 5 cm, and a thickness of about 0.1 mm to about 0.5 mm.

5. The device of claim 1, wherein the substructure is a face mask; and the sorptive tab is mounted on the inside surface of the face mask facing the subject.

6. The device of claim 1, wherein the substructure is a bubble wand, stick, or forceps.

7. The device of claim 1, wherein the sorptive tab further comprises a nucleic acid or an antibody.

8. The device of claim 1, wherein the sorptive tab is removably mounted on the substructure by a staple.

9. A method, comprising the steps of: (1) obtaining a sample of breath aerosol from a subject using a device of claims 1; and (2) analyzing the sample for the presence or absence of a target analyte.

10. The method of claim 9, wherein the target analyte is an analyte for detecting a disease, a drug, health, a metabolite, a nucleic acid, a protein, a microorganism, a virus, or exposure to an environmental chemical.

11. The method of claim 9, wherein
the target analyte is a target analyte for disease detection or the target analyte is a target analyte for disease exclusion; and
the target analyte for disease detection is present or the target analyte for disease exclusion is absent, thereby indicating that the subject is a diseased subject; and
the method further comprises the step of: (3) administering to the subject a treatment for a detected disease.

12. The method of claim 9, wherein the substructure is a face mask; and the sorptive tab is mounted on the inside surface of the face mask facing the subject.

13. The method of claim 12, wherein the face mask is positioned over the nose or mouth of the subject to obtain the sample.

14. The method of claim 9, wherein the substructure is a bubble wand, stick, or forceps.

15. The method of claim 14, wherein the sorptive tab is positioned in front of the nose or mouth of the subject.

16. The method of claim 9, further comprising the step of: removing the sorptive tab from the substructure after the sample is obtained.

17. The method of claim 9, wherein the sample is analyzed by mass spectrometry.

18. The method of claim 9, wherein the sample is analyzed by cation mass tag mass spectrometry.

19. The method of claim 9, wherein the sample is analyzed by nucleic acid hybridization, nucleic acid sequencing, or immunoassay.

20. The method of claim 9, wherein the sorptive tab further comprises a nucleic acid or an antibody.
